(19) United States
(12) Patent Application Publication
(10) Pub. No.: US 2016/0309068 A1
(43) Pub. Date: Oct. 20, 2016

(54) SPATIAL FREQUENCY DOMAIN IMAGING USING CUSTOM PATTERNS

(71) Applicant: THE REGENTS OF THE UNIVERSITY OF CALIFORNIA, Oakland, CA (US)

(72) Inventors: Kyle Nadeau, Irvine, CA (US); Tyler B. Rice, Irvine, CA (US); Soren Konecky, Irvine, CA (US); Anthony J. Durkin, Irvine, CA (US); Bruce J. Tromberg, Irvine, CA (US)

(73) Assignee: The Regents of the University of California, Oakland, CA (US)

(21) Appl. No.: 15/108,271

(22) PCT Filed: Jan. 5, 2015
(86) PCT No.: PCT/US2015/010201

§ 371 Date: Jun. 24, 2016

Related U.S. Application Data
(60) Provisional application No. 61/924,151, filed on Jan. 6, 2014.

(51) Int. Cl. H04N 5/225; A61B 1/06; A61B 1/00; G01N 21/64; A61B 5/026; A61B 1/04; A61B 5/00; G01J 3/28; G01B 11/25; A61B 5/1455

(52) U.S. Cl. CPC H04N 5/2256; G01B 11/2513; A61B 1/0669; A61B 1/00009; A61B 5/1455; A61B 5/0261; A61B 1/043; A61B 1/0684; A61B 5/6826; G01J 3/2846; G01N 21/6456; G01N 21/6486

(57) ABSTRACT

The present invention relates to optical devices and methods of extracting optical properties, and depth and fluorescence information for visualizing samples. In one embodiment, the present invention provides a multi-frequency synthesis and extraction (MSE) method for quantitative tissue imaging. In another embodiment, the present invention provides a method of obtaining optical properties and depth information by illuminating a sample with binary square wave patterns of light, wherein a series of spatial frequency components are simultaneously attenuated and can be extracted. In another embodiment, the present invention provides an optical imaging apparatus comprising a Spatial Frequency Domain Imaging (SFDI) device modified to condense frequency information content into a single charged coupled device (CCD) frame, multi-pixel and/or single-pixel sensor using frequency-synthesized patterns.

### GOVERNMENT RIGHTS

This invention was made with Government support under Grant No. RR001192, awarded by the National Institutes of Health. The Government has certain rights in this invention.

### FIELD OF THE INVENTION

The invention relates to the field of optics and more specifically, detection of spatial frequency components.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

There are various optical imaging tools and methods that may be used in conjunction with biomedical diagnostics and treatments. For example, Diffuse Optical Spectroscopic Imaging (DOSI) is a technique that can quantify absorption and scattering coefficients of tissues up to several centimeters deep. Or, for example, SFDI (Spatial Frequency Domain Imaging) is a quantitative optical imaging modality that employs spatially-modulated to separate light scattering from absorption in its measurements. Unlike DOSI, SFDI is a wide-field optical technique, and works by taking advantage of the Fourier inverse of point source-detector measurements by projecting light into spatially sinusoidal patterns onto a sample such as a tissue sample. In turn, absorption and scattering quantification can give information about the sample, where by analyzing the spatial modulation transfer function for the diffusion of light within the tissue, both depth and quantifiable optical properties can be extracted for various practical applications. However, currently available optical imaging techniques are also not without their limitations and disadvantages. For example, limited speed is an issue in SFDI, where there is a need for multiple frames of data, and there are difficulties in increasing data acquisition speed to the frame-rate of a camera. Thus, there is a need in the art for more effective optical imaging devices and methods.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts, in accordance with embodiments herein, graphs demonstrating that SFDI works by taking advantage of the Fourier inverse of point source-detector measurements by projecting light into spatially sinusoidal patterns onto a tissue sample. FIG. 1(a) depicts sinusoidal pattern projection of light onto tissue and simulated cross-sectional view of photon density. Low spatial frequencies blur more slowly and therefore penetrate more deeply into tissue. FIG. 1(b) depicts sample plot of the reflectance as a function of spatial frequency k. The shape of the curve depends on the optical properties of the sample.

FIG. 2 depicts, in accordance with embodiments herein, examples of propagation of patterned light through turbid media. The projected image and corresponding Fourier Transform (FT) is shown. The tissue attenuates the spatial frequencies, acting as a high pass filter, and the resulting image appears blurred.

FIG. 3 depicts, in accordance with embodiments herein, a stack of 100 images of custom projected disks at different radii. The attenuation of the Fourier spectrum of the disk was determined and used to fit for the amount of absorption (top) and scattering (bottom). These matched expected values to within 10%.

FIG. 4 depicts, in accordance with embodiments herein, simulation of 1D cross-section of square wave pattern (50% duty cycle) interacting with turbid media. After interacting with a sample, the edge of the square wave is blurred in space. As a result, each frequency component is simultaneously attenuated.

FIG. 5 depicts, in accordance with embodiments herein, results demonstrating the inventors' MSE. (a) Simulated workflow of multi-frequency synthesis and extraction (MSE) algorithm on a turbid sample containing a circular absorbing lesion. First, a square wave (duty cycle=50%) and a DC (planar) image are acquired. Here, the low-pass filtering properties of the sample damp the higher-ordered harmonics of the square wave, such that only the fundamental frequency component is preserved. These images are applied to a 2D Hilbert transform method, from which the phase angle map of the square wave pattern is derived. Using the known Fourier series representation of a square wave (Eq 3), the frequency coefficient matrix (Ck) is generated. Finally, Ck is inverted and multiplied by the raw data vector (I). (b) Extracted spatial frequency intensities from simulation shown in (a), including (i) DC and (ii) fundamental frequencies. (iii) Cross-section of extracted reflectance comparing MSE to conventional, 3-phase SFDI.

FIG. 6 depicts, in accordance with embodiments herein, absorption and reduced scattering (μa and μs′) maps generated using (a) conventional SFDI (sinusoidal patterns) and (b) multi-frequency synthesis and extraction (MSE) approaches, using 3 phase-offset square wave patterns at a wavelength of 659 nm. Here we see agreement in μa and μs′ values in the region of interest (ROI, black box) within 0.3 and 0.8% for μa and μ′s, respectively.

FIG. 7 depicts, in accordance with embodiments herein, in vivo forearm results using square wave patterns and multi-frequency synthesis and extraction. (MSE). (a) Reflectance maps extracted at DC, fundamental (0.06 mm-1), 2nd, and 3rd harmonic frequencies. (b) Mean raw and calibrated reflectance values from forearm ROI (black box) at DC, fundamental, 2nd, and 3rd harmonics. Absorption and (d) reduced scattering maps derived using diffusion model fit and DC, fundamental, and 2nd harmonic reflectance maps, with mean values within 0.8% and 0.2%, respectively.

FIG. 8 depicts, in accordance with embodiments herein, multi spatial frequency reflectance results obtained on a phantom containing a slanted absorbing tube ranging in depth from 0 to 54 mm, containing an absorbing dye with a scattering background 1%. (a) Reflectance maps calibrated to a homogenous tissue-simulating phantom are derived for DC (0 mm-1), 0.06, 0.09, 0.12, and 0.18 mm-1 using the fundamental and 2nd harmonic components from two square wave patterns. (b) Cross-sections of calibrated reflectance taken from horizontal line in center of image where tube is located. Mean reflectance values along the line agree to within 0.2%, 2.3%, 1.4%, 1.1%, and 2.9% for 0, 0.06, 0.09, 0.12, and 0.18 mm-1 respectively.

FIG. 9 depicts, in accordance with embodiments herein, a flowchart for data acquisition and processing using the multi-frequency synthesis and extraction (MSE) technique. This approach allows for the extraction of images of multiple spatial frequency components using custom projection patterns.

FIG. 10 depicts, in accordance with embodiments herein, SFDI instrument using custom, multi-frequency patterns. Modulation hardware is highly versatile, and may consist of the following: Electronic spatial light modulator (SLM) such as digital micromirror device (DMD) (in binary mode, DMD's can run 1-2 orders of magnitude faster than grayscale mode (i.e. sinusoids)); Transmission/reflection mask (Spiral pattern, checkerboard pattern (rotating or laterally shifting)); Physical objects such as fan; Light sources having spatial patterns (LED array (circles), scanning laser line, etc.). Unconventional modulation hardware such as physical objects have no refresh rate, so images can be acquired at the minimum exposure time of the camera.

FIG. 11 depicts, in accordance with embodiments herein, a schematic of an endoscope embodiment using custom projection patterns. The benefit here with using custom patterns as opposed to sinusoids is that low resolution waveguides such as fiber bundles can be used to transport the pattern from the modulator (at other end of the endoscope) to the sample. Binary patterns such as dots or lines do not require high spatial resolution to project. Also, having the modulator and light source placed outside of the scope allows for greater hardware versatility and reduced endoscope footprint.

FIG. 12 depicts, in accordance with embodiments herein, results using multi-frequency synthesis standalone. (a) Optical property results obtained using spatial frequency information content derived from applying custom, multi-frequency pattern to tissue-simulating phantom. The difference in mean absorption (μa) from the ROI (shown in b) between conventional, 3-phase SFDI and synthesis is 0.00%, while the difference in reduced scattering (μs′) was 0.12% (1.1221 vs. 1.1208 mm-1).

FIG. 13 depicts, in accordance with embodiments herein, simulation data combining the Hilbert and synthesis techniques. (a) Simulated intensity images taken at three phases using a custom, multi-frequency pattern with spatial frequencies of 0, 0.05, 0.15, and 0.25 mm-1 with intensities of 6, 1, 2, and 3 respectively, with a field-of-view of approximately 10×10 cm. (b) Additional, phase-shifted images derived from applying images in (a) to the Hilbert method. (c) Extracted reflectance maps derived from applying images from (a) and (b) to the synthesis technique. The reflectance maps corresponding to 0.05 (top) and 0.15 mm-1 (middle) show reflectance values that are within 2% of the expected value for most pixels, and the map for 0.25 mm (bottom) has reflectance values within 1% of the expected value for most pixels.

FIG. 14 depicts, in accordance with embodiments herein, a schematic of transmission geometry SFDI instrument using multi-frequency projection patterns. A projector (e.g. DMD, mechanical object, LED array) projects light onto the sample. Here, a sample having cm scale thickness (e.g. mouse) is imaged, which is typically not possible in reflection mode. In this configuration, detected light travels further compared to reflection mode. Therefore, the s-MTF of the sample is lower, and thus lower frequency square wave patterns can be employed. Additionally, tomographic reconstruction is possible by analyzing the attenuated frequency components in the multi-frequency pattern.

FIG. 15 depicts, in accordance with embodiments herein, a schematic of transmission, ring-based SFDI instrument. Here, custom patterns are projected using a small form-factor SLM such as an LED array. The spatial frequency components from the pattern are attenuated as the light is absorbed and scattered by the sample. The transmitted light is detected by a CCD or photodiode array. Similar to FIG. 14, in another embodiment, tomographic reconstruction is possible. In another embodiment, this instrument could be implemented in a watch form factor.

### SUMMARY OF THE INVENTION

Various embodiments include a method of obtaining optical data from a sample, comprising illuminating a sample with multi-frequency patterns having arbitrary spatial frequency intensities, and extracting one or more images of multiple spatial frequency components. In another embodiment, illuminating the sample comprises illuminating the sample with binary patterns of light. In another embodiment, illuminating the sample comprises use of an electronic spatial light modulator. In another embodiment, illuminating the sample comprises moving a mechanical object. In another embodiment, moving a mechanical object includes rotating and/or moving laterally. In another embodiment, moving a mechanical object includes use of a physical shape. In another embodiment, the physical shape includes one or more of varying spiral, fan blade and checkerboard shapes. In another embodiment, the method further comprises use of a patterned light source. In another embodiment, the patterned light source includes an LED array. In another embodiment, the patterned light source includes a line-scanning laser. In another embodiment, the number of spatial frequency components extracted from the pattern is limited to an equivalent number of required frames. In another embodiment, the pattern is phase shifted for each frame taken. In another embodiment, each spatial frequency component in each frame is mapped. In another embodiment, each frame is mapped by use of a 2D Hilbert transform technique. In another embodiment, each frame is mapped by projecting an additional pattern to calibrate location of a single phase. In another embodiment, each frame is mapped by treating phase angle as an additional parameter in a matrix equation herein. In another embodiment, the method further comprises inputting data into a multi-frequency synthesis and extraction (MSE) matrix inversion algorithm to determine the demodulated reflectance for each spatial frequency component. In another embodiment, the method further comprises obtaining sensitivity to superficial layers and/or scatterings from the sample by utilizing the fundamental component from higher frequency binary patterns. In another embodiment, the method further comprises obtaining probing of deep layers from the sample by utilizing the fundamental component from lower frequency binary patterns. In another embodiment, the method further comprises SFD tomography. In another embodiment, the method further comprises 3D reconstructions. In another embodiment, the method further comprises a combination of multiple frequency components extracted from a low-frequency pattern and fundamental components extracted from a high-frequency pattern. In another embodiment, the sample is a biological sample or tissue. In another embodiment, the sample is a human forearm. In another embodiment, the method further comprises quantitative analysis of the sample. In another embodiment, the quantitative analysis of the sample includes quantitative analysis of tissue composition and/or changes in composition. In another embodiment, the extracted images of multiple spatial frequency components are part of a multi-spectral, video-rate Spatial Frequency Domain Imaging (SFDI) system. In another embodiment, the extracted images of multiple spatial frequency components are made in conjunction with a scientific-grade CMOS (sCMOS) camera. In another embodiment, the extracted images of multiple spatial frequency components are made in conjunction with a digital imaging sensor. In another embodiment, the digital imaging sensor includes a camera phone. In another embodiment, the digital imaging sensor is a single element detector. In another embodiment, the digital imaging sensor is a photodiode in a compressive sensing (CS) configuration. In another embodiment, the extracted images of multiple spatial frequency components are detected by a spectrometer. In another embodiment, multiple AC, non-planar spatial frequency components are extracted from the sample simultaneously. In another embodiment, the sample is in vivo tissue. In another embodiment, the sample is an organism. In another embodiment, the sample is a plant. In another embodiment, the sample is physically part of an individual. In another embodiment, the sample is a turbid medium. In another embodiment, the method is a component of a burn wound triage protocol. In another embodiment, the method is a component of a skin cancer screening protocol. In another embodiment, the method is performed in conjunction with reconstructive and/or general surgery.

Other embodiments include a data and processing apparatus, comprising a device adapted for illuminating a sample with a binary pattern followed by a quantitative analysis of the sample. In another embodiment, the sample is a turbid medium. In another embodiment, the apparatus further comprises a projection pattern. In another embodiment, the projection pattern is carried by a low resolution waveguide. In another embodiment, the projection pattern is carried in free space. In another embodiment, the projection pattern is carried in a high resolution waveguide. In another embodiment, the projection pattern is carried in a liquid core light guide. In another embodiment, the projection pattern is carried by a fiber bundle. In another embodiment, the device is an endoscope. In another embodiment, the endoscope has a light source located outside of the scope component of the endoscope. In another embodiment, the device is adapted to extract one or more images of multiple spatial frequency components. In another embodiment, the device is a Spatial Frequency Domain Imaging (SFDI) system comprising a structured light illumination system configured to condense frequency information content into a frame using frequency-synthesized patterns. In another embodiment, the quantitative analysis of the sample further comprises extracting images of multiple spatial frequency components. In another embodiment, quantitative analysis of the sample includes a multi-frequency synthesis and extraction (MSE) method. In another embodiment, the apparatus further comprises Spatial Frequency Domain Spectroscopy (SFDS). In another embodiment, the quantitative analysis of the sample includes fluorescence detection capabilities.

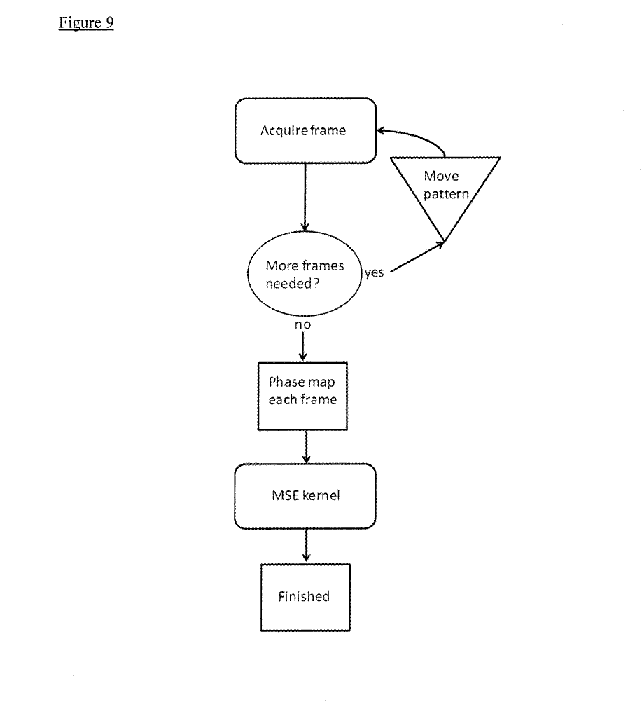

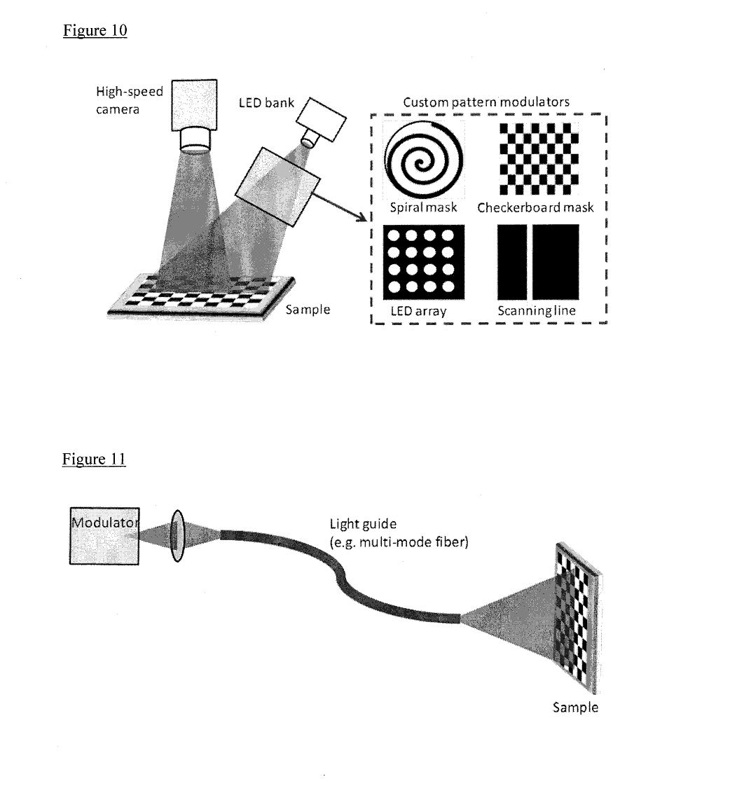

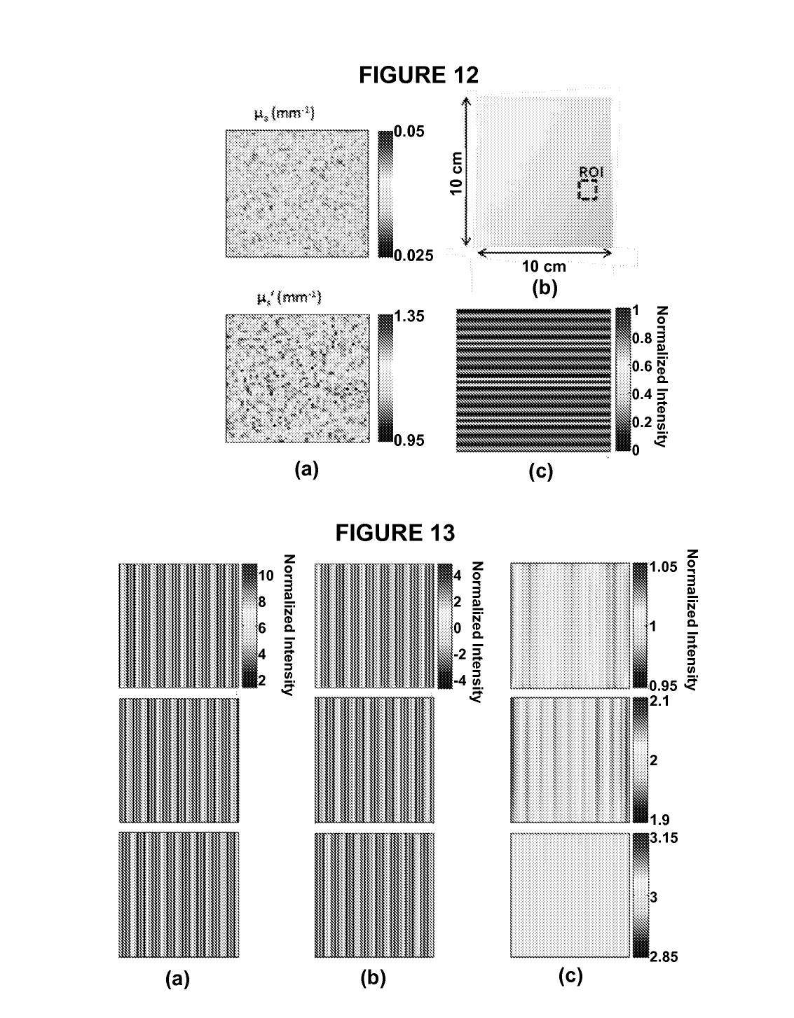

Other embodiments include an optical imaging apparatus, comprising a structured light illumination system configured to condense frequency information content into a frame using frequency-synthesized patterns. In another embodiment, the structured light illumination system is a Spatial Frequency Domain Imaging (SFDI) system. In another embodiment, the sample is a turbid medium. In another embodiment, the frame is part of a single Charged Coupled Device (CCD). In another embodiment, the frame is part of a multi-pixel sensor array. In another embodiment, the apparatus further comprises an NIR light source homogenized through an integrating rod and/or sent through a mechanical projecting device. In another embodiment, the mechanical projecting device is a motorized expanding disk, non-expanding disk, fan shape, expanding ring, and/or non-expanding ring. In another embodiment, the apparatus further comprises an electronic spatial light modulator. In another embodiment, the apparatus further comprises a transmission and/or reflectance mask. In another embodiment, the apparatus further comprises a light source with a spatial pattern. In another embodiment, the apparatus further comprises a Spatial Frequency Domain Spectroscopy (SFDS). In another embodiment, the apparatus further includes fluorescence detection capabilities. In another embodiment, the apparatus is described in FIG. 10 herein. In another embodiment, the apparatus is described in FIG. 11 herein.

Various embodiments include a method of imaging tissue, comprising visualizing and/or projecting a tissue sample of a subject through an optical imaging apparatus comprising a structured illumination device configure to condense frequency information content into a frame using frequency-synthesized patterns. In another embodiment, the structured illumination device is a Spatial Frequency Domain Imaging (SFDI) device. In another embodiment, the sample is a turbid medium. In another embodiment, the frame is part of a single Charged Coupled Device (CCD). In another embodiment, the frame is part of a multi-sensor pixel array. In another embodiment, the optical imaging apparatus may be used to analyze physical properties of the tissue. In another embodiment, physical properties includes chemical properties. In another embodiment, the data acquisition speed is increased to the frame rate of a camera by using patterns. In another embodiment, there is no projector chip.

Other embodiments include a method of diagnosing a disease in a subject, comprising analyzing the physical properties of a sample from a subject using an optical imaging apparatus comprising a structured illumination device configured to condense frequency information content into a single frame using frequency-synthesized patterns, and diagnosing the disease based on the physical properties of the sample. In another embodiment, the structured illumination device is a Spatial Frequency Domain Imaging (SFDI) device. In another embodiment, the single frame is a single Charged Coupled Device (CCD) frame. In another embodiment, the physical properties of the sample include tissue biological function. In another embodiment, the physical properties of the sample include hemodynamics and/or chemical constituents. In another embodiment, the subject is human. In another embodiment, the subject is an organism. In another embodiment, the subject is a plant. In another embodiment, the sample is a turbid medium.

Other embodiments include a method of prognosing a disease and/or predicting health in a subject, comprising analyzing the physical properties of a sample from a subject using an optical imaging apparatus comprising a structured illumination device configured to condense frequency information content into a frame using frequency-synthesized patterns, and determining the severity of a disease and/or predicting sample health based on the physical properties of the sample. In another embodiment, the structured illumination device is a Spatial Frequency Domain Imaging (SFDI) device. In another embodiment, the frame is part of a single Charged Coupled Device (CCD) frame. In another embodiment, the frame is part of a multi-pixel sensor array. In another embodiment, the physical properties of the sample include tissue biological function at high temporal resolution, including hemodynamics and chemical constituents. In another embodiment, the method further comprises analysis of time to heal from the disease. In another embodiment, the subject is human. In another embodiment, the method further comprises treatment of the disease. In another embodiment, the sample is a turbid medium.

Various embodiments include a method of obtaining optical properties, and depth and fluorescence information, comprising illuminating and/or receiving from a sample multi-frequency patterns having arbitrary spatial frequency intensities, and extracting a single pixel image of one or more spatial frequency components. In another embodiment, the multi-frequency patterns comprises a binary square wave pattern of light using a projection pattern. In another embodiment, the sample is a turbid medium.

Other embodiments include a data and processing apparatus, comprising a device adapted for transmission of a sample with a binary pattern followed by a quantitative analysis of the sample. In another embodiment, the transmission includes transmission of neutrons. In another embodiment, the transmission includes transmission of X-Rays. In another embodiment, quantitative analysis of the sample includes fluorescence detection capabilities. In another embodiment, the sample is a turbid medium.

Other embodiments include a method of evaluating tissue health in a subject, comprising analyzing tissue from a subject using an optical imaging apparatus comprising a structured illumination device configured to condense frequency information content into a frame using frequency-synthesized patterns to analyze the physical properties of the sample, and evaluating tissue health based on the physical properties of the tissue. In another embodiment, the structured illumination device is a Spatial Frequency Domain Imaging (SFDI) device. In another embodiment, the sample is a turbid medium. In another embodiment, the physical properties of the tissue include one or more of tissue biological function, chemical function, and structure. In another embodiment, the frame is part of a single Charged Coupled Device (CCD) frame. In another embodiment, the frame is part of a multi-pixel sensor device. In another embodiment, the frame is part of a single-pixel sensor device. In another embodiment, the method further comprises SFD tomography. In another embodiment, multi-frequency information may be extracted to generate a 3D reconstruction. In another embodiment, the method is described in FIG. 9 herein.

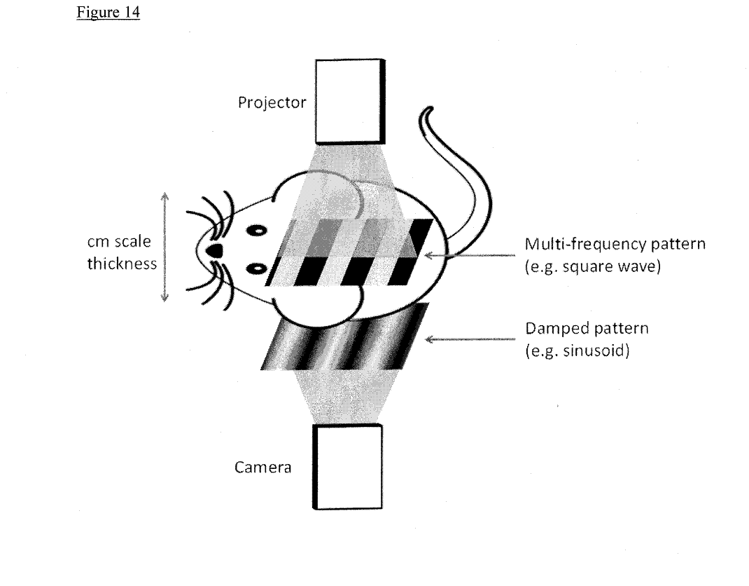

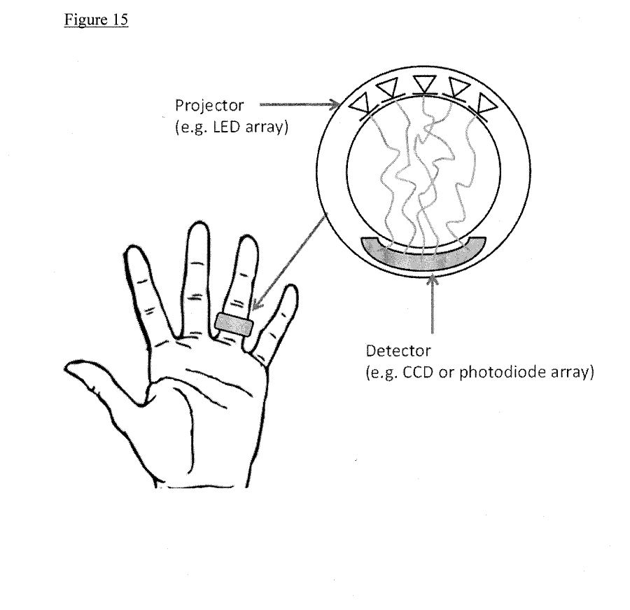

Various embodiments also include an apparatus, comprising a transmission geometry instrument using multi-frequency patterns. In another embodiment, the instrument is described in FIG. 14 herein. In another embodiment, the instrument is described in FIG. 15 herein.

Other features and advantages of the invention will become apparent from the following detailed description, taken in conjunction with the accompanying drawings, which illustrate, by way of example, various embodiments of the invention.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Brady et al., Optical Imaging and Spectroscopy, Wiley-OSA (2009); Hornyak, et al., Introduction to Nanoscience and Nanotechnology, CRC Press (2008); Singleton et al., Dictionary of Microbiology and Molecular Biology 3rd ed., J. Wiley & Sons (New York, N.Y. 2001); and Advanced Organic Chemistry Reactions, Mechanisms and Structure 7th ed., J. Wiley & Sons (New York, N.Y. 2013), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

References hereby incorporated by reference include and are not limited to the following: Duarte, et al., “Single-pixel imaging via compressive sampling,” IEEE Signaling Processing Magazine, March 2008; Saager, et al., “Determination of optical properties of turbid media spanning visible and near-infrared regimes via spatially modulated quantitative spectroscopy,” Journal of Biomedical Optics 15(1), January/February 2010; and Konecky, et al., “Quantitative optical tomography of sub-surface heterogeneities using spatially modulated structured light,” Optics Express, Vol. 17, No. 17, Aug. 5, 2009.

As used herein, the abbreviation “SFDI” means Spatial Frequency Domain Imaging.

As used herein, the abbreviation “CCD” means Charged Coupled Device.

As used herein, the abbreviation “MSE” means multi-frequency synthesis and extraction.

As disclosed herein and in accordance with various embodiments herein, the inventors have developed a multi-frequency synthesis and extraction (MSE) method for quantitative tissue imaging. In one embodiment, by illuminating a sample with binary square wave patterns of light, a series of spatial frequency components are simultaneously attenuated, and can be extracted to determine optical property and depth information. Additionally, binary patterns are projected much faster than sinusoids that are typically used in spatial frequency domain imaging (SFDI), allowing for short (millisecond or less) camera exposure times. Spatial frequency component intensity maps are determined by acquiring frames of square wave reflectance data at unique phases. In another embodiment, these data are then applied to a matrix inversion algorithm which resolves each spatial frequency component pixel-by-pixel. The inventors compared optical property and depth penetration results extracted using square waves to those obtained using single frequency sinusoidal patterns on an in vivo human forearm and absorbing tube phantom, respectively. Absorption and reduced scattering coefficient values were shown to agree to within 1% using both single and multiple AC frequencies, and depth penetration reflectance values agree to within 1%. In another embodiment, the combined use of MSE with square wave patterns allow for the development of a multi-spectral, video-rate SFDI instrument.

As further described herein, the quantity of absorption and scattering properties in tissue is determined by projecting light with customized structure, and measuring attenuation of the Fourier spatial frequency components. In accordance with various embodiments herein, the inventors developed optical imaging that does not require the relatively slow projection of 3 phase offsets at each spatial frequency of conventional SFDI. Rather, the inventors have packed all frequency information content into a single CCD frame using frequency synthesized patterns.

In one embodiment, the present invention provides an optical imaging apparatus comprising a Spatial Frequency Domain Imaging (SFDI) device modified to condense frequency information content into a single CCD frame using frequency-synthesized patterns. In another embodiment, the present invention further comprises an NIR light source homogenized through an integrating rod and/or sent through a mechanical projecting device. In another embodiment, the mechanical projecting device is a motorized expanding disk, fan shape, and/or expanding ring.

In another embodiment, the present invention provides a method of imaging tissue, comprising providing an optical imaging apparatus comprising a Spatial Frequency Domain Imaging (SFDI) device modified to condense frequency information content into a single CCD frame using frequency-synthesized patterns, and visualizing and/or projecting a tissue sample of a subject through the optical imaging apparatus. In another embodiment, the optical imaging apparatus may be used to analyze physical properties of the tissue. In another embodiment, the data acquisition speed is increased to the frame rate of a camera by using custom patterns and with no projector chip.

In accordance with various embodiments herein, the present invention provides an apparatus of optical imaging where absorption and scattering quantification provide information about biological function in a subject, including the diagnosis of a disease, prognosis of a disease and/or healing response. In another embodiment, data acquisition speed is increased, such as to the frame rate of a camera, by using custom patterns where one can project simple shapes such as a disk or ring, using physical objects and optics, and with no projector chip. In another embodiment, the present invention provides a technique for imaging biological function at high temporal resolution, such as hemodynamics and chemical constituents.

In one embodiment, the present invention provides a method of diagnosing a disease in a subject, comprising providing a sample from a subject, using an optical imaging apparatus comprising a Spatial Frequency Domain Imaging (SFDI) device modified to condense frequency information content into a single CCD frame using frequency-synthesized patterns to analyze the physical properties of the sample, and diagnosing the disease based on the physical properties of the sample. In another embodiment, the physical properties of the sample include tissue biological function at high temporal resolution, including hemodynamics and chemical constituents. In another embodiment, the subject is human.

In another embodiment, the present invention provides a method of diagnosing susceptibility to a disease in a subject, comprising providing a sample from a subject, using an optical imaging apparatus comprising a Spatial Frequency Domain Imaging (SFDI) device modified to condense frequency information content into a single CCD frame using frequency-synthesized patterns to analyze the physical properties of the sample, and diagnosing susceptibility to the disease based on the physical properties of the sample. In another embodiment, the physical properties of the sample include tissue biological function at high temporal resolution, including hemodynamics and chemical constituents. In another embodiment, the subject is human.

In another embodiment, the present invention provides a method of prognosing a disease in a subject, comprising providing a sample from a subject, using an optical imaging apparatus comprising a Spatial Frequency Domain Imaging (SFDI) device modified to condense frequency information content into a single CCD frame using frequency-synthesized patterns to analyze the physical properties of the sample, and prognosing a severe form of the disease based on the physical properties of the sample. In another embodiment, the physical properties of the sample include tissue biological function at high temporal resolution, including hemodynamics and chemical constituents. In another embodiment, the method further comprises analyzing time to heal from the disease in the subject. In another embodiment, the subject is human.

As further disclosed herein, the inventors have developed methods and devices for data acquisition and processing using a multi-frequency synthesis and extraction (MSE) technique. In one embodiment, this approach allows for the extraction of images of multiple spatial frequency components using custom projection patterns. For example, use of a patterned light source can eliminate the need for SLM, and decrease instrument complexity.

In one embodiment, the patterns are generated by an electronic spatial light modulator. In another embodiment, the electronic spatial light modulator is a DMD. In another embodiment, the patterns are generated by a moving mechanical object. In another embodiment, the moving mechanical object moves by rotation and/or movement laterally. In another embodiment, the moving mechanical object includes shapes such as spiral, fan blade, and/or checkerboard. In another embodiment, the patterns are generated by a patterned light source. In another embodiment, the patterned light source is a LED array. In another embodiment, the patterned light source is a line-scanning laser.

As further disclosed herein, in one embodiment, the custom pattern is projected onto a sample and frames of data are acquired. The minimum number of frames required is equivalent to the number of spatial frequency components extracted from the pattern (for example, 3 frames for 3 spatial frequencies). For each frame taken, the pattern should be phase-shifted or “moved.”

As further disclosed herein, in another embodiment, the phases for each spatial frequency component in each frame are mapped. Once the raw data is acquired and phase maps are determined, this information is input to the MSE matrix inversion algorithm, which determines the demodulated reflectance for each spatial frequency component described in the matrix herein. In one embodiment, each spatial frequency component in each frame may be mapped using a 2D Hilbert transform approach. In another embodiment, each spatial frequency may be mapped by projecting an additional pattern to calibrate location of a single phase. In another embodiment, projecting an additional pattern to calibrate location of a single phase may be accomplished by a thin line in center of field of view and/or single sinusoid. In another embodiment, each spatial frequency component in each frame may be mapped by treating phase angle as an additional parameter and solving in the matrix equation, requiring an additional frame for each spatial frequency component.

In another embodiment, a single element detector may be used, such as a photodiode in a compressive sensing (CS) configuration, for example. In another embodiment, the CS configuration may employ binary patterns to encode 2D images in a 1D time array. In another embodiment, the invention includes the utilization of the binary patterns intrinsic to CS instruments by superimposing these patterns on top of CS patterns. Or, for example, in another embodiment, a spectrometer could be used for detection. In another embodiment, light remitted from the sample from a broadband source is coupled to a spectrometer from a single pixel, which divides the light into multiple spectral components at a single point in space. Since MSE processes data on a pixel-by-pixel basis, it will be possible to analyze data taken from a single point in space.

In another embodiment, the present invention provides for SFD tomography, where a combination of multiple frequency components extracted from a low-frequency pattern and the fundamental component(s) extracted from a high-frequency pattern may be used to do a 3D reconstruction.

In another embodiment, fluorescence information may be obtained. As fluorescence may be light emitted from a sample, for example, fluorescence information may be obtained in conjunction with various embodiments herein as it's characteristics are similar to reflected (or transmitted) light such that MSE may be applied in the same manner.

In another embodiment, the present invention may be used in a transmission geometry configuration. For example, with a transmission geometry instrument, light can be detected from deeper depths compared to reflectance geometry. In another embodiment, the spatial frequency components in these detected patterns would be attenuated more than those detected in reflectance mode, which could enable isolation of the fundamental component from lower frequency patterns. In accordance with various embodiments herein, a transmission geometry set up may include: 1.) small animal imager where the projected light passes through the entire animal before detection, and 1) “iWatch” or ring type device.

As readily apparent to one of skill in the art, the technique is in no way limited to binary patterns, and information and/or data may be obtained, for example, using multi-frequency sinusoidal patterns (i.e. patterns containing a superposition of single-frequency sinusoids). In another embodiment, the present invention includes multi-frequency patterns having arbitrary spatial frequency intensities.

Further, as used herein, the term “sample” is not in any way only limited to biological samples that are taken from and analyzed apart from an individual. A sample may include, for example, a target to be analyzed and/or visualized while it is still part of a living individual, such as visualizing and/or analyzing a body part such as an arm, or muscle tissue, of an individual.

As readily apparent to one of skill in the art, various embodiments herein as relating to methods of extracting one or more spatial frequency components for optical imaging may be used in conjunction with any number of devices and related methods. For example, in one embodiment, the present invention provides an optical device utilizing a single pixel detector. In another embodiment, the optical device is part of a compressive sensing (CS) and/or spatially modulated quantitative spectroscopy (SMoQS) setup. Or, for example, the invention may relate to the ability to process fluorescence information. Or, for example, as further described herein, the invention may also be used for a transmission geometry configuration, and is in no way limited to reflectance. In another embodiment, the present invention provides a device for SFD tomography, where multi-frequency information may be extracted to generate, for example, 3D reconstructions of absorbers, fluorophores, and/or scatterers.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Custom Pattern SFDI

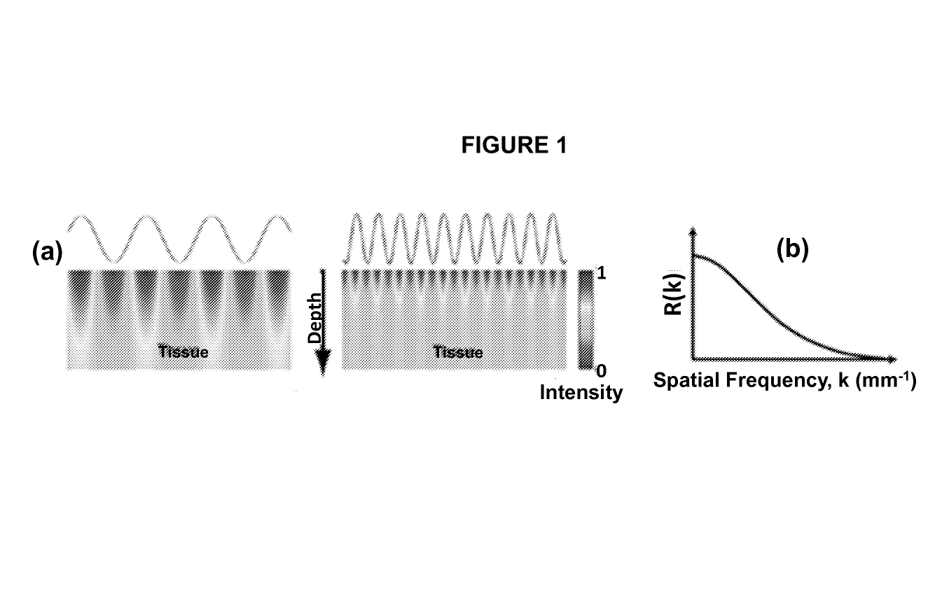

SFDI works by taking advantage of the Fourier inverse of point source-detector measurements by projecting light into spatially sinusoidal patterns onto a tissue sample (FIG. 1(a)). The inventors' general modeling framework is based on the time independent diffusion approximation to light transport:

∇²φ−μ_{eff}(τ)²φ=S(r)∇²φ−μ_{eff}(τ)²φ=S(r) (1)

where φ is the light fluence, μₜᵣ=μₐ+μₛ′, and μ_{eff}=(3μₐμₜᵣ′)^{1/2} Here S(r) is the light source term. Traditionally this equation is solved analytically for a delta function light source (point-like). However, if S(r) is written as a sinusoidal intensity wave with frequency k:

S(r)=I₀ cos(kx+θ) (2)

S(r)=A*cos (kx+θ) The fluence can be solved for analytically as a function of spatial frequency and depth. By applying the partial current boundary condition, the reflectance can be solved for as a function of spatial frequency k

$$R ( k ) = \frac{3 A {μ}_{s}^{′} \text{/} {μ}_{tr}}{( \frac{{μ}_{{eff}^{( k )}}^{′}}{{μ}_{tr}} + 1 ) ( \frac{{μ}_{{eff}^{( k )}}^{′}}{{μ}_{tr}} + 3 A )} \qquad ( 3 )$$

where μ_{eff}(k)=(3μₐμₜᵣ′+k²)^{1/2}. Here A accounts for the index of refraction mismatch A=(1−R_{eff})/[2(1+R_{eff})]. A plot of what R(k) looks like for a given set of optical properties can be seen in FIG. 1(b) herein.

Note that R(k) acts as a low-pass filter and is a nonlinear function of spatial frequency, absorption, and scattering, which can be fit with a minimum of two data points. In practice, the source will have some DC offset. What actually exits the sample is Iₘₑₐₛ=R(0)*I₀+R(k)*I₀*cos(kx+θ). To extract only the remitted amplitude of the projected sine wave R(k), a demodulation scheme is employed. If waves are projected with 3 phase offsets separated by 2π/3 radians, the amplitude can be solved for:

$$R ( k ) = \frac{\sqrt{2}}{3} [ {( {I}_{1} - {I}_{2} )}^{2} + {( {I}_{1} - {I}_{3} )}^{2} + {( {I}_{2} - {I}_{3} )}^{2} ] \\ R = \frac{\sqrt{2}}{3} [ {( {I}_{1} - {I}_{2} )}^{2} + {( {I}_{1} - {I}_{3} )}^{2} + {( {I}_{2} - {I}_{3} )}^{2} ] \qquad ( 4 )$$

This conventional approach provides a robust method for SFDI measurements of tissue optical properties. However, a broad goal is to develop a new method that does not require the relatively slow projection of 3 phase offsets at each spatial frequency. Rather, they pack all frequency information content into a single CCD frame using frequency-synthesized patterns. Herein lies the difficulty in analyzing arbitrary projected patterns and shapes: no analog to demodulation.

To begin solving this issue, the inventors first write the source and reflectance in terms of its fourier series.

Iᵢₙ=S(r)=Σₙ₌₀^{N}Σₘ₌₀^{M}C(kₓⁿ,k_{y}^{m})exp[i*(kₓⁿx+k_{y}^{m}y)]

Iᵢₙ=S(r)=Σₖₓ^{N}Σ_{ky}^{M}C(kₓ,k_{y})exp[i*(kₓx+k_{y}y)] (5)(a)

Iₒᵤₜ=Σₙ₌₀^{N}Σₘ₌₀^{M}R(|k|)C(kₓⁿ,k_{y}^{m})exp[i*(kₓⁿx+k_{y}^{m}y)] (5)(b)

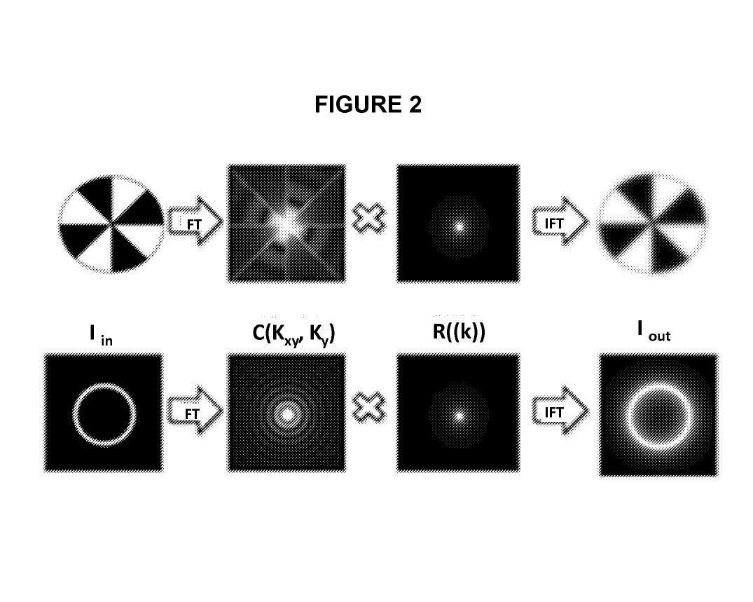

Determining the function R(|k|) will allow for the fitting of optical properties. The Fourier coefficients C are known analytically for many simple shapes (such as a disk or ring) but can also be found numerically for complex patterns using a Discrete Fourier Transform (DFT). One example of what the forward process might look like for two simple shapes, a fan and ring, is given in FIG. 2 herein.

Once C is found and the remitted light Iₒᵤₜ is measured, R(|k|) becomes the only unknown. To solve for R(|k|) algebraically, as many equations as |k| values are needed. Thus to manipulate the shape of the projection a total of P times, each of which will have a different Fourier series. Then, R(|k|) can be solved for using simple linear algebra

$$\begin{matrix}( \begin{matrix}{I}_{1} ( x , y ) \\ ⋮ \\ {I}_{P} ( x , y )\end{matrix} ) = ( \begin{matrix}{C}_{1} ( {k}_{x}^{1} , {k}_{y}^{1} ) {e}^{- i ( {k}_{x}^{1} x + {k}_{y}^{1} y )} & … & {C}_{1} ( {k}_{x}^{N} , {k}_{y}^{M} ) {e}^{- i ( {k}_{x}^{N} x + {k}_{y}^{M} y )} \\ ⋮ & ⋱ & ⋮ \\ {C}_{P} ( {k}_{x}^{1} , {k}_{y}^{1} ) {e}^{- i ( {k}_{x}^{1} x + {k}_{y}^{1} y )} & … & {C}_{P} ( {k}_{x}^{N} , {k}_{y}^{N} ) {e}^{- i ( {k}_{x}^{N} x + {k}_{y}^{M} y )}\end{matrix} ) * ( \begin{matrix}R ( \vert {k}_{11} \vert ) \\ ⋮ \\ R ( \vert {k}_{NM} \vert )\end{matrix} ) \\ ( \begin{matrix}{I}_{1} \\ ⋮ \\ {I}_{P}\end{matrix} ) = ( \begin{matrix}{C}_{1} ( {k}_{1} , {k}_{1} ) {e}^{- i ( {k}_{1} x + {k}_{1} y )} & … & {C}_{1} ( {k}_{N} , {k}_{M} ) {e}^{- i ( {k}_{N} x + {k}_{M} y )} \\ ⋮ & ⋱ & ⋮ \\ {C}_{P} ( {k}_{1} , {k}_{1} ) {e}^{- i ( {k}_{1} x + {k}_{1} y )} & … & {C}_{P} ( {k}_{N} , {k}_{M} ) {e}^{- i ( {k}_{N} x + {k}_{M} y )}\end{matrix} ) * ( \begin{matrix}R ( \vert {k}_{11} \vert ) \\ ⋮ \\ R ( \vert {k}_{NM} \vert )\end{matrix} ) & ( 6 ) ( a ) \\ I = C * R ⇒ R = {C}^{- 1} * I \\ I = C * R \to R = {C}^{- 1} * I & ( 6 ) ( b )\end{matrix}$$

For non-square matrices, C⁻¹ will be the pseudo-inverse. Once the reflectance is found as a function of spatial frequency at each pixel, standard optical property mapping can be used. These include: analytic function fits to diffusion solutions (eq. 1), Monte Carlo (MC) simulations, and rapid lookup-table approaches.

A NIR light source (broadband and/or discrete LED sources) is homogenized through an integrating rod, and sent through a mechanical projection device. This may be a motorized expanding disk, fan shape (shown), expanding ring, or other pattern. The optimal projection structure will be evaluated as part of the proposal before this aspect of the device is installed.

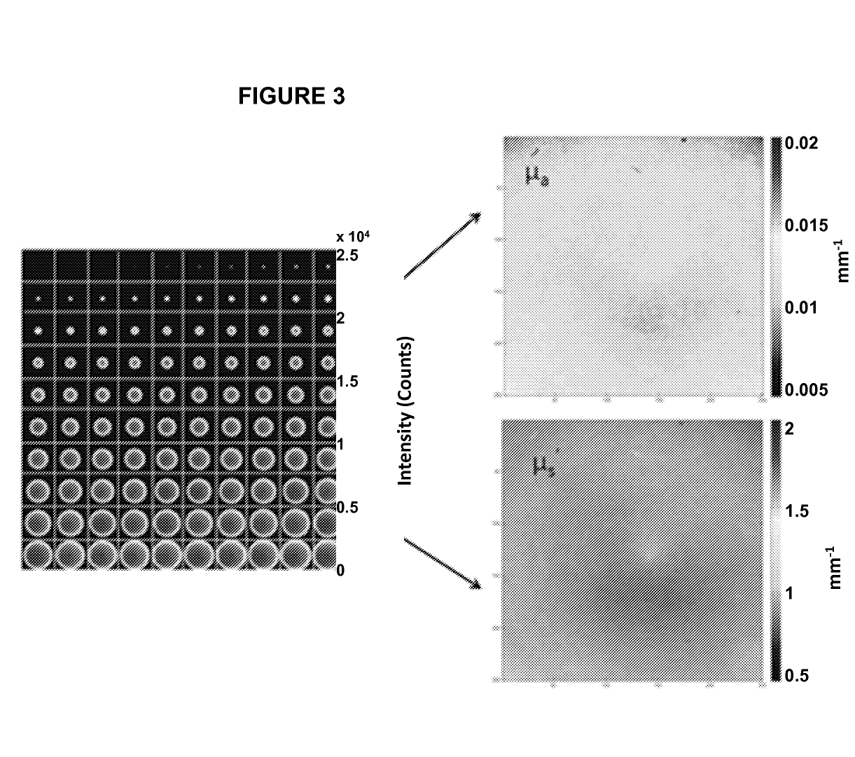

Research proves the viability of this method. FIG. 3 herein shows intensity images of 100 disks of different radii projected onto a silicone tissue simulating phantom, and the extracted optical properties utilizing the methods above.

### Example 2

### Overall-MSE for Quantitative Imaging

As further disclosed herein, a method for high-speed spatial frequency domain (SFDI) data acquisition, utilizing a multi-frequency synthesis and extraction (MSE) method and binary, square wave projection patterns for quantitative tissue imaging. Spatial frequency component intensity maps are determined by acquiring frames of square wave reflectance data at unique phases. These data are then applied to a matrix inversion algorithm which resolves each spatial frequency component pixel-by-pixel. By illuminating a sample with binary square wave patterns of light, a series of spatial frequency components are simultaneously attenuated, and can be extracted to determine optical property and depth information. Additionally, binary patterns are projected faster than sinusoids that are typically used in spatial frequency domain imaging (SFDI), allowing for short (millisecond or less) camera exposure times, and thus data acquisition speeds an order of magnitude or more greater faster than conventional SFDI. In cases where sensitivity to superficial layers or scattering is important, the fundamental component from higher frequency square wave patterns can be used. When probing deeper layers is critical, the fundamental and harmonic components from lower frequency square wave patterns can be used. The inventors compared optical property and depth penetration results extracted using square waves to those obtained using single frequency sinusoidal patterns on an in vivo human forearm and absorbing tube phantom, respectively. Absorption and reduced scattering coefficient values are shown to agree to within 1% using both single and multiple AC frequencies, and depth penetration reflectance values agree to within 1%. The combined use of MSE with square wave patterns allow for the development of a multi-spectral, video-rate SFDI instrument.

### Example 3

### Background

The analysis of light propagation in the spatial frequency domain allows for the quantitative analysis of biological tissue. The relationship that governs this analysis is known as the spatial modulation transfer function (s-MTF). The s-MTF states that the attenuation of spatial photon density waves in turbid media depends on the wave's frequency and the sample's absorption and scattering properties. It has been previously reported the use of spatial frequency domain analysis for tissue optical property (i.e. absorption and reduced scattering coefficient) extraction. The inventors employed a radially-varying square wave pattern, applying one dimensional Fourier transforms to a cross-section of the pattern, and utilized the intensity value corresponding to the DC (planar illumination) and fundamental frequency components. In this case, optical properties are determined at a point in space. Others have developed an alternate method using 2D frequency domain analysis of pulse train patterns, resulting in the mapping of the integrated s-MTF over a wide frequency band, and the derivation of the spatial mean s-MTF curve for the entire image. Similarly, others have developed spatial frequency domain imaging (SFDI), which employs sinusoidal patterns to extract single frequency spatial frequency reflectance maps. The s-MTF is then fit to these maps pixel-by-pixel, resulting in images of absorption (μa) and reduced scattering (μs′) coefficients.

Conventional SFDI encodes individual spatial frequency components into each frame by illuminating the sample with DC-offset sinusoidal patterns. SFDI systems typically use digital micromirror devices (DMD's) to project light onto the sample, whose mirror array elements flicker on and off several times to generate grayscale intensities, resulting in a maximum pattern refresh rate typically on the order of single milliseconds. High-end scientific-grade CMOS (sCMOS) cameras have the ability to acquire frames on the order of kHz or greater, exceeding the grayscale pattern refresh rate of DMD's, resulting in an SFDI data acquisition bottleneck. In many cases, such as those where the sample is susceptible to motion artifacts or fine temporal dynamics are being probed, data acquisition speed is critical. Additionally, certain applications require multiple spatial frequency components, most notably SFD tomography, which relies on the spatial frequency dependence of depth penetration in turbid media. Ideally, multiple AC (non-planar) spatial frequency components could be extracted from a sample simultaneously, although this not possible using sinusoidal patterns. Binary patterns such as square waves have the potential to increase SFDI data acquisition speed by an order of magnitude or greater. Square waves patterns require only a single on/off state for each pixel, and thus can be generated on the order of hundredths of milliseconds, roughly two orders of magnitude faster than sinusoids. Additionally, square waves contain frequency components at the even and odd harmonics of the fundamental frequency that can be synthesized into each SFDI frame, increasing the amount of spatial frequency information embedded into each frame of data.

As further described herein, an aim of the inventors was to increase SFDI data acquisition speed by an order of magnitude or greater by using square wave patterns. To accomplish this, in accordance with various embodiments herein, they developed a multi-frequency synthesis and extraction (MSE) algorithm. Custom patterns having known Fourier series coefficients are applied to the sample. The sample acts as a filter, characterized by the sample's s-MTF, attenuating the spatial frequency components of the diffusely reflected light. By changing the amplitude and phase of the Fourier series components of the pattern, a system of equations is established for each Fourier component at each pixel in the image, and can be solved using a pseudoinverse. MSE has the flexibility of extracting an arbitrary number of frequency components from a sample. If a single (or higher) AC (modulated) frequency is required, a square wave having a higher fundamental frequency can be used such that the higher ordered terms are highly attenuated by the sample. If multiple (or lower) AC frequencies are required, a square wave having a lower fundamental frequency can be used, such that higher ordered Fourier terms are preserved. Additionally, MSE adapts to sample height and topography by employing a 2D phase angle mapping approach based on the Hilbert Transform.

Agreement is shown between the new MSE technique and conventional SFDI by comparing μa and μs′ maps derived using a DC and single AC (fundamental) component, and multiple (DC, fundamental, and 2nd harmonics) extracted from an in vivo human forearm. Also demonstrated are multi-frequency depth penetration results on a phantom containing a buried absorbing tube surrounded by turbid medium. The results described herein demonstrate that SFDI data acquisition speed for mapping μa and μs′ and probing buried inclusion (SFD tomography) can be increased by an order of magnitude or greater with minimal losses in data quality using square wave patterns and MSE.

### Example 4

### Multi Spatial Frequency Synthesis and Extraction Using Square Wave Patterns for Quantitative Tissue Imaging

Spatial Frequency Domain Imaging (SFDI):

The SFDI workflow, including data acquisition, processing, and analysis have been previously disclosed. First, sinusoidal patterns are projected onto a sample, and a camera detects the remitted light at the sample boundary, whose spatial frequency constituents have been damped due to the absorption and scattering properties of the sample. In conventional SFDI, sinusoidal patterns having a single modulation frequency at 3 distinct phases are detected and applied to a simple demodulation formula based on square-law detection shown in Eq. 1. Data is also taken on a phantom having known optical properties, which is used to normalize the sample intensity to account for the transfer function of the SFDI instrument. Finally, the calibrated reflectance data at multiple spatial frequencies is used to derive the sample's s-MTF, from which optical property maps are determined.

$$AC ( x , y ) = \frac{{2}^{1 \text{/} 2}}{3} \{ [ {R}_{0 °} ( x , y ) - {{R}_{120 °} ( x , y ) ]}^{2} + {{[ {R}_{120 °} ( x , y ) - {R}_{240 °} ( x , y ) ]}^{2} + {[ {R}_{240 °} ( x , y ) - {R}_{0 °} ( x , y ) ]}^{2} \}}^{1 \text{/} 2} \qquad ( Eq . 1 )$$

### Where

R_{φ}(x,y)=½+½ sin(ω_{x,y}+φ)

Where ω is the angular frequency

Conventional SFDI requires that sinusoidal patterns are used, and thus spatial frequency data is acquired sequentially. As further disclosed herein, the inventors provide MSE, a new method for capturing the s-MTF of biological tissue that makes use of rapidly-generated binary square wave patterns containing multiple spatial frequency components.

Multi Frequency Synthesis and Extraction (MSE) Technique:

A goal of MSE is to use custom patterns having multiple spatial frequency components, and extract the attenuated spatial frequency components remitted from the sample. First, the inventors' acquire a set of images having different pattern phases. One can express the series of raw intensity images as a vector (I), which is the product of the Fourier series coefficients of each frame with the reflectance at each spatial frequency component, shown in Eq. 2. Here, C represents the frequency amplitude and phase maps for each projected pattern. For consistency, we express each frequency component as a real-valued sinusoid, although single complex exponentials (analytical expression) could also be used. R represents the amplitude attenuation for each frequency component in the reflectance maps. k and p are the indices for projected pattern and Fourier component, and m and n are the total number of projected patterns and Fourier components, respectively.

$$\begin{matrix} {I}_{p} ( x , y ) = {C}_{k , p} ( x , y ) * {R}_{k} ( x , y ) \to {R}_{k} ( x , y ) \\ = {{C}_{k , p} ( x , y )}^{- 1} * {I}_{p} ( x , y )\end{matrix} \\ {I}_{p} ( x , y ) = [ \begin{matrix}{I}_{1} ( x , y ) \\ ⋮ \\ {I}_{m} ( x , y )\end{matrix} ] \\ {R}_{k} ( x , y ) = [ \begin{matrix}{R}_{1} ( x , y ) \\ ⋮ \\ {R}_{n} ( x , y )\end{matrix} ] \\ {C}_{k , p} ( x , y ) = [ \begin{matrix}{C}_{1 , 1} ( x , y ) sin ( \begin{matrix}{ω}_{1} ( x , y ) + \\ {ϕ}_{1} ( x , y )\end{matrix} ) & … & {C}_{1 , n} ( x , y ) sin ( \begin{matrix}{ω}_{n} ( x , y ) + \\ {ϕ}_{1} ( x , y )\end{matrix} ) \\ ⋮ & ⋱ & ⋮ \\ {C}_{m , 1} ( x , y ) sin ( \begin{matrix}{ω}_{1} ( x , y ) + \\ {ϕ}_{m} ( x , y )\end{matrix} ) & … & {C}_{m , n} ( x , y ) sin ( \begin{matrix}{ω}_{n} ( x , y ) + \\ {ϕ}_{m} ( x , y )\end{matrix} )\end{matrix} ] \qquad ( Eq . 2 )$$

Where k and p are the indices for projected pattern and Fourier component, and m and n are the total number of projected patterns and Fourier components, respectively

In principle, this approach can be applied to any multi-frequency pattern, assuming that the phase and amplitude of each frequency component are known. The inventors are employing binary square wave patterns for the aforementioned projection speed benefit. In general, a square wave pattern in one dimension can be expressed by a Fourier series, shown in Eq. 3. Here, d is the duty cycle of the square wave, denoted as the fraction of high to low intensity values, and ω and φ are the angular frequency and phase, respectively. This is an infinite series, however, the low-pass filter nature of biological tissue eliminates higher-ordered harmonics, which can be neglected, assuming they have been sufficiently damped relative to the previous terms in the Fourier series.

$$\begin{matrix}SquareWave ( x ) = \frac{4}{π} {\sum }_{k = 1}^{∞} ( \frac{1}{k} ) cos ( π kd ) sin ( ω kx + ϕ ) \\ = \frac{4}{π} [ cos ( π kd ) sin ( ω x + ϕ ) + \\ \frac{cos ( π kd )}{2} sin ( 2 ω x + ϕ ) + \\ \frac{cos ( π kd )}{2} sin ( 3 ω x + ϕ ) + … ]\end{matrix} \qquad ( Eq . 3 )$$

As further disclosed herein, the inventors present a cross-section of a square wave pattern. The duty cycle of the pattern can be adjusted to change the Fourier coefficients of each harmonic component. After interacting with a sample, the edges of the pattern are blurred, and thus the pattern appears more sinusoidal. In reality, a combination of multiple frequency components are embedded into the pattern.

To extract information from a given pattern using MSE, the amplitude and phase of each frequency component in the pattern must be determined. The amplitude coefficients are known from the analytical expression of the pattern itself. However, deriving the phase is non-trivial. For one, the position of the phase of the pattern field generated will most likely not match what the camera detects. For example, the camera requires a field of view that is smaller than the projected pattern, and thus the field of view of the camera will not match that of the projected pattern. Second, depending on the angle of the camera relative to the projected patterns, sample topography will affect phase angle. The inventors previously developed a technique using a variant of a 2D Hilbert transform to express SFDI images in their analytical form, from which amplitude and phase angle maps can be determined. The phase maps generated using the Hilbert technique also adapt to surface topography. The inventors have integrated the phase mapping capability of the Hilbert technique into MSE.

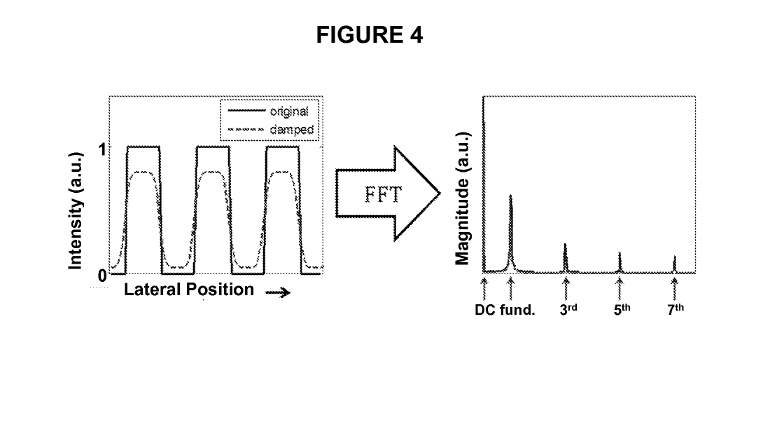

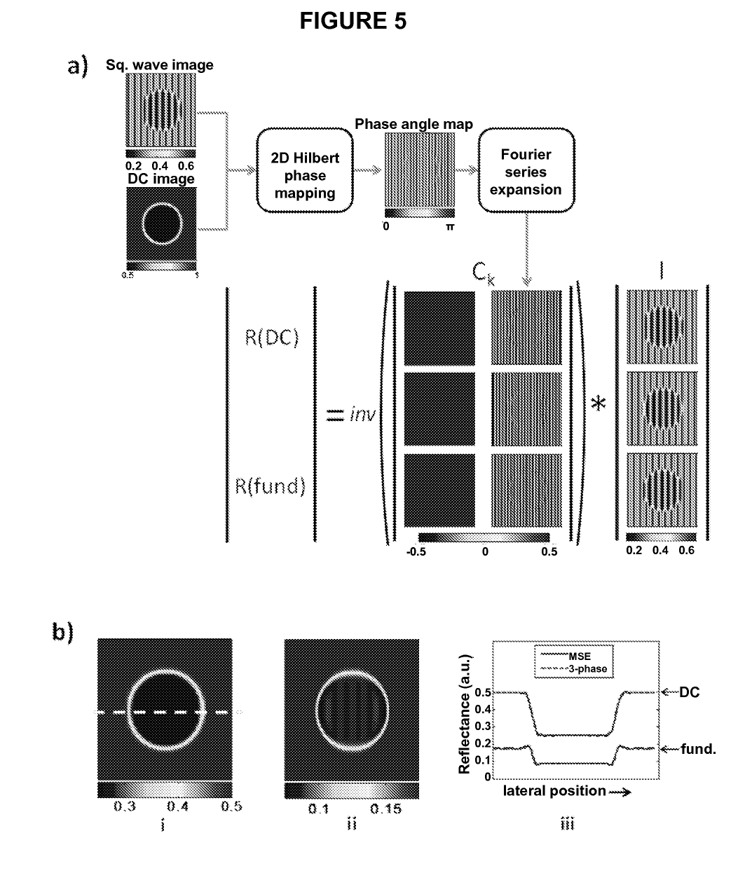

To demonstrate the technique, FIG. 4 herein shows results on a simulated sample consisting of an absorbing lesion and a uniform scattering background. This is the simplest case of MSE where the sample filters out all frequency components in the square wave except for the fundamental. A damped square wave image, which appears sinusoidal, and a DC image are applied to the Hilbert technique to extract a phase angle map. Next, phase map coefficients are derived by using the phase angle map is used in conjunction with the square wave Fourier series expansion. Finally, the Fourier coefficient matrix C is inverted and multiplied by the raw intensity vector I to obtain the reflectance vector R.

For simplicity, FIG. 4 herein illustrates MSE for a damped square wave pattern having harmonic components which surpass the s-MTF limit of the sample, such that only a single frequency component remains in the reflected light. However, MSE has the flexibility to extract multiple spatial frequency components from a pattern. As further described herein, the inventors demonstrate that μa and μs′ can be accurately determined on an in vivo forearm using a square wave pattern by extracting DC and fundamental frequency components. Also shown are μa and μs′ results using a lower fundamental frequency square wave pattern, from which three spatial frequency components (DC, fundamental, and 2nd, harmonic) are extracted and used to determine μa and μs′. Finally, the inventors exhibit the ability for MSE to extract data capable of layered or tomographic reconstruction by measuring reflectance vs. depth using two square wave patterns having different fundamental frequencies, and extracting DC, fundamental, and 2^{nd} harmonic components from each.

Results:

Fitting to the s-MTF requires a minimum of two spatial frequencies, which are used to decouple μs′ from μa. In the simplest case, one can employ a DC (planar) and a single AC (modulated) component to derive ua and us′ maps, which is demonstrated in the 1st experiment below using a square wave pattern at a relatively high fundamental spatial frequency. In the 2nd experiment below, the inventors demonstrate how multiple AC frequency components can be extracted from a sample using a single pattern at several phases having a relatively low fundamental spatial frequency. The inventors then use this data to map μa and μs′. In the 3rd experiment below, the inventors show reflectance maps taken from a phantom consisting of a buried absorbing tube occupying a range of depths surrounded by a background of 1% Intralipd. To generate the data used to produce the images analyzed in this section, the inventors employed a 2nd generation clinical SFDI (VIS-NIR, Modulated Imaging Inc., Irvine, Calif.) All data processing and computation used to produce figures was performed using the MATLAB software suite (MATLAB and Statistics Toolbox Release 2012b, The MathWorks, Inc., Natick, Mass.).

High Spatial Frequency In Vivo Optical Property Extraction:

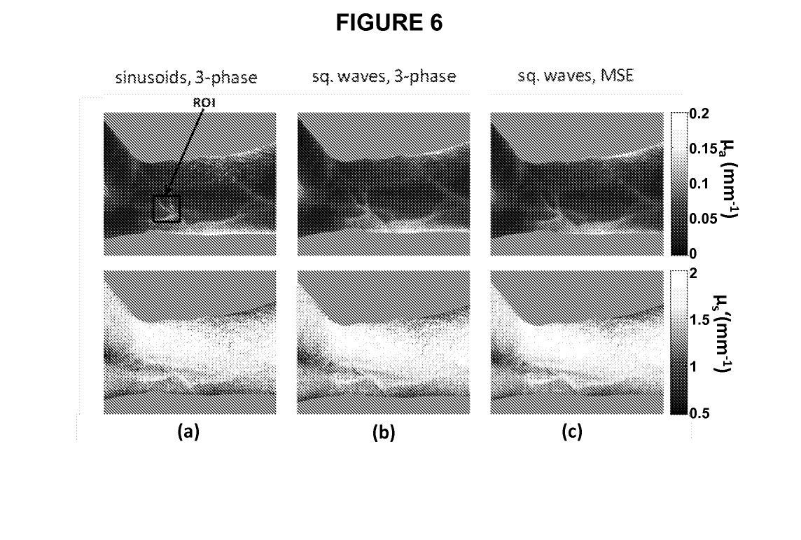

They performed a side-by-side comparison of optical property maps derived using two spatial frequency components extracted using conventional, 3-phase demodulation (Eq. 1) and MSE at a modulation frequency of 0.28 mm-1, and a source wavelength of 659 nm, shown in FIG. 6 herein. For conventional SFDI, 3 phase-offset sinusoidal patterns (Eq. 1) and a DC frame are taken to extract the DC and AC spatial frequency components. For MSE, 3 phase-offset square wave patterns with a duty cycle of 50% are taken. The 50% duty cycle was chosen to maximize the separation between the fundamental and nearest harmonic component to allow for optimal damping damping, since 50% duty cycle square waves have no even (i.e. 2nd) harmonic components.

FIG. 6 herein shows agreement in optical property values to within 1% for both pa and μs′ using sinusoidal and square wave illumination with 3-phase demodulation (Eq. 1) and MSE, respectively. These findings imply that, for the simplest case of SFDI where only a DC and single AC component are used, that square wave patterns can be employed instead of sinusoids. The reason for this is because the higher ordered terms in the square wave pattern (i.e. 3rd, 5th, 7th etc. harmonics) are highly attenuated relative to the fundamental term, and thus can be neglected in the MSE phase mapping and inversion algorithm. In this case, the conventional 3-phase demodulation equation (Eq 1) can be used since the pattern appears sinusoidal. In FIG. 6 herein, the inventors decouple the effects of the square wave damping from MSE by demonstrating that both 3-phase demodulation and MSE are accurate in deriving optical properties on the same dataset.

It should be noted that, for square wave patterns to produce high quality data, the choice of spatial frequency is non-trivial. In the case above, the inventors chose a fundamental frequency such that all of the higher-ordered harmonics are essentially eliminated due to the filtering properties of the sample. They found that, for most biological samples, a 50% duty cycle square wave with a fundamental frequency less than 0.25 mm-1 will generate higher ordered terms. In this case, the next (3rd order) term is attenuated by roughly and order of magnitude. Additionally, since square wave patterns have only 2 unique intensity values (off or on), the number of projector pixels used to generate a single period of the pattern must be even (for 50% duty cycle), such that the duty cycle is consistent. The pixel length of the projected square wave period should also be divisible by the number of phases used to avoid duty cycle changes from phase-shifting the pattern.

To fully utilize MSE, the remitted light in the raw data images may contain multiple spatial frequency components. This is possible because the MSE inversion algorithm accounts for an arbitrary number of spatial frequency components, whereas the 3-phase approach (Eq. 1) relies on sinusoidal patterns (1 AC spatial frequency component). They show that this is possible in the next experiment using a pattern having a relatively low fundamental frequency, such that a subset of the higher-ordered harmonic terms are preserved, and can thus be factored into the inversion algorithm and extracted.

Multiple AC Frequency In Vivo Optical Property Extraction:

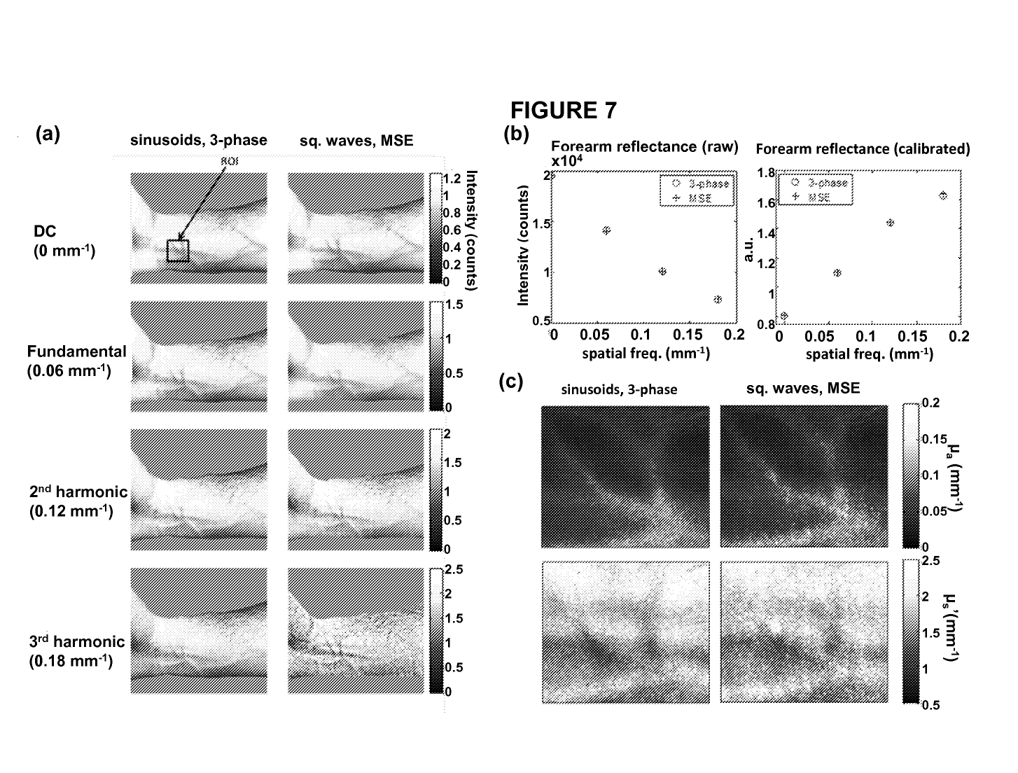

In a similar manner to the previous experiment, the inventors compared optical property maps derived using MSE to 3-phase demodulation. However, instead of taking DC and a single AC frequency component, here they extract DC and 3 AC frequencies. The advantage here is that the accuracy of optical property fitting increases, since there are more s-MTF points along which to fit. These results are shown in FIG. 7 herein, where 7 uniformly phase-offset square wave patterns having a fundamental frequency of 0.06 mm-1 and a duty cycle of 75% are used to extract the DC, fundamental, 2^{nd} and 3rd harmonic components, corresponding to 0, 0.06, 0.12, and 0.18 mm-1, respectively. The 0, 0.06, and 0.12 mm-1 component maps are applied to a diffusion model, from which μa and μs′ maps are derived. These optical property values are compared directly to those obtained using 3-phase demodulation and sinusoidal patterns at the same spatial frequencies.

FIG. 7 herein shows that higher ordered harmonics can be extracted using a single multi-frequency square wave pattern, and that optical property values agree with those obtained using conventional, 3-phase demodulation and single-frequency sinusoidal patterns using a diffusion model. The use of multiple AC spatial frequency components increases the accuracy of optical property mapping, and thus quantitatition of chromophore concentrations and structural parameters. Being able to access multiple frequency components quickly using a single pattern will reduce the data acquisition burden associated with obtaining multiple frequency components.

It should be noted that the presence of noise and artifacts related to higher-ordered harmonics increases with the higher-ordered terms (i.e. 3rd harmonic in this case). This is due to the fact that the higher-ordered terms in a square wave pattern have less intensity relative to the higher-ordered terms, compared to the DC, fundamental, and 2nd harmonic terms. Additionally, since the phase angle of the sinusoidal terms cannot be extracted from the square wave pattern directly, we currently use sinusoidal patterns to determine phase angle.

The mean interrogation depth of SFDI patterns in biological tissue is dependent on the spatial frequency component; lower spatial frequencies penetrate deeper while higher spatial frequencies probe more superficial layers. Thus, 3D reconstruction is possible by extracting and analyzing multiple spatial frequency components in SFDI. For these reconstructions to be accurate, each individual spatial frequency component must interrogate the appropriate depths. In the following experiment below, the inventors show that MSE and multi-frequency square wave patterns give similar reflectance maps compared to those derived using 3-phase SFDI on a buried absorbing tube phantom.

Depth Penetration Experiment Using Buried Absorber Phantom:

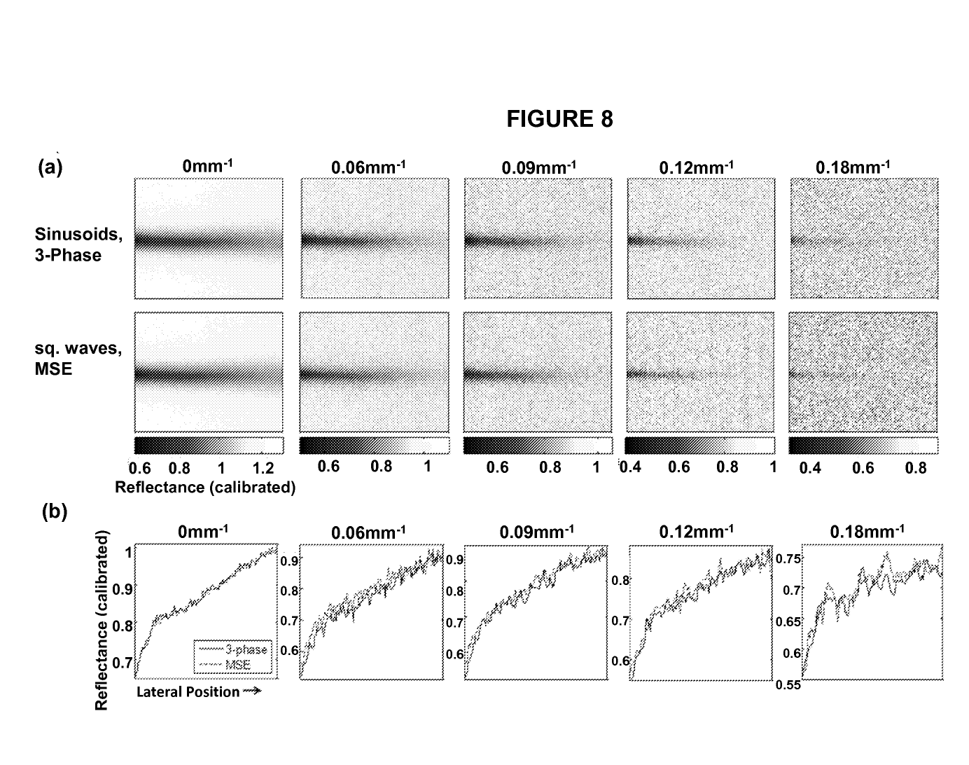

Multiple SFDI spatial frequency components can be extracted and processed to perform 3D reconstructions of buried absorbers. To accurately pinpoint these inclusions in depth, it is critical that the spatial frequency components interrogate the appropriate depths in the sample. To test the ability to extract the correct depth information using square wave patterns, they applied MSE to a depth phantom containing a buried absorbing tube oriented diagonally, such that the depth of the tube ranges from 0 to 4 mm beneath the surface. The tube contains a solution of 1% Intralipid and 0.5 g/L of a NIR absorbing dye (NIR746A, QCR Solutions Corp., Fort St. Lucie, Fla.), which was chosen to closely match the μa of venous blood. The background contains a 1% solution of Intralipid. FIG. 8 herein shows results comparing reflectance maps taken at 731 nm using 3-phase demodulation and MSE. Similar to the previous experiment, 7 phases of a pattern having a fundamental frequency of 0.07 mm-1 and a duty cycle of 75% are taken. The calibrated reflectance is extracted for DC, fundamental, and 2nd harmonic components.

The results shown in FIG. 8 herein indicate that the multi spatial frequency component extracted using MSE and square wave patterns yield depth penetration reflectance similar to 3-phase demodulation using single frequency patterns for the DC, fundamental, and 2nd harmonic components. This implies that MSE can be used to extract multi-frequency datasets, which could be applied to SFD tomography, since reflectance maps at multiple spatial frequencies are what are used to reconstruct buried absorbers.

SFDI has the ability to provide information-rich datasets based on the acquisition and analysis of spatial frequency domain reflectance maps. However, data acquisition speed should ideally be limited to the camera frame rate, and sinusoidal projection patterns used in conventional SFDI take significantly longer to project than the exposure times of most high-end, scientific-grade cameras. As disclosed herein, the inventors have created a new signal processing technique, MSE, allowing for the extraction of SFD information content using square wave patterns, which can be generated faster than frame rates of current high-end cameras.

The results demonstrate that μa and μs′ maps derived using MSE and square wave patterns yield results similar to conventional SFDI (>1% difference). In the 1st experiment, they exhibited the low-pass filter characteristics of tissue, by applying a square wave pattern whose higher-ordered harmonic components are virtually eliminated. The fundamental component is left intact, and the reflectance information contained in this pattern (DC and AC) is extracted. In the 2nd experiment, they use a square wave pattern having a relatively low fundamental spatial frequency, such that the higher-ordered harmonic components can be utilized. Here they derived reflectance maps at DC, fundamental, 2nd, and 3rd harmonics, and computed optical property maps that agree to with conventional SFDI using the same frequencies to within 1%. In this 3rd experiment, they applied the same square wave pattern scheme to a phantom containing a buried. slanted absorbing tube having a continuum of absorber depths as a function of lateral spatial location. Here, reflectance values obtained along the tube at multiple spatial frequencies using MSE agree with conventional SFDI, implying that SFD tomography is possible.

MSE can accommodate spatial patterns having arbitrary spatial frequency components. In principle, any SFDI pattern could be applied to a sample and analyzed using MSE. Thus, one may use alternate spatial light modulators (SLM's), whose patterns contain multiple spatial frequency components. A rotating fan, for example, contains radially-varying square wave patterns. Such a device would be far less costly than a DMD, for example, and would have no refresh rate. Alternatively, a light source having intrinsic spatial frequency patterns such as an LED array could be employed, eliminating the need for an SLM.

The higher ordered harmonics in a square wave pattern are attenuated more than lower ordered components. Additionally, biological tissue naturally attenuate higher ordered terms more due to scattering. Thus, in order to maximize the signal-to-noise ratio of these higher-ordered terms, the detector used should have high dynamic range. Single element detectors (SED's) such as photodiodes are both cost-effective and highly sensitive/dynamic ranges. Detectors such as SED's could be used instead of cameras to give the sensitivity and dynamic range needed to extract additional higher-ordered spatial frequency terms from MSE patterns.

In conclusion, the inventors developed, described and demonstrated a new technique (MSE) for extracting images of multiple spatial frequency components using square wave patterns of structured light. This method employs a matrix inversion algorithm by mapping the phase and amplitude of each frequency component embedded into the pattern, and multiplying the Fourier coefficient matrix by the raw intensity images pixel-by-pixel. By using square wave patterns, multiple spatial frequency components are simultaneously extracted, and SFDI data acquisition speed is potentially increased by an order of magnitude or greater. They have applied MSE to an in vivo forearm model and a depth penetration phantom, from which optical property and reflectance maps were derived, respectively, showing agreement to conventional, 3-phase SFDI. The use of binary patterns and MSE in the SFDI workflow will allow for increased data acquisition speeds and new spatial light modulators which will drive down costs and footprint of future SFDI instruments.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A method of obtaining optical data from a sample, comprising:
illuminating a sample with multi-frequency patterns having arbitrary spatial frequency intensities; and
extracting one or more images of multiple spatial frequency components.

2. The method of claim 1, wherein illuminating the sample comprises illuminating the sample with binary patterns of light.

3. The method of claim 1, wherein illuminating the sample comprises use of an electronic spatial light modulator.

4. The method of claim 1, wherein illuminating the sample comprises moving a mechanical object.

5. The method of claim 4, wherein moving a mechanical object includes rotating and/or moving laterally.

6. The method of claim 4, wherein moving a mechanical object includes use of a physical shape.

7. The method of claim 6, wherein the physical shape includes one or more of varying spiral, fan blade and checkerboard shapes.

8. The method of claim 1, further comprising use of a patterned light source.

9. The method of claim 8, wherein the patterned light source includes an LED array.

10. The method of claim 8, wherein the patterned light source includes a line-scanning laser.

11. The method of claim 1, wherein the number of spatial frequency components extracted from the pattern is limited to an equivalent number of required frames.

12. The method of claim 11, wherein the pattern is phase shifted for each frame taken.

13. The method of claim 1, wherein each spatial frequency component in each frame is mapped.

14. The method of claim 13, wherein each frame is mapped by use of a 2D Hilbert transform technique.

15. The method of claim 13, wherein each frame is mapped by projecting an additional pattern to calibrate location of a single phase.

16. The method of claim 13, wherein each frame is mapped by treating phase angle as an additional parameter in a matrix equation herein.

17. The method of claim 1, further comprising inputting data into a multi-frequency synthesis and extraction (MSE) matrix inversion algorithm to determine the demodulated reflectance for each spatial frequency component.

18. The method of claim 1, further comprising obtaining sensitivity to superficial layers and/or scatterings from the sample by utilizing the fundamental component from higher frequency binary patterns.

19. The method of claim 1, further comprising obtaining probing of deep layers from the sample by utilizing the fundamental component from lower frequency binary patterns.

20. The method of claim 1, further comprising SFD tomography.

21. The method of claim 1, further comprising 3D reconstructions.

22. The method of claim 21, further comprising a combination of multiple frequency components extracted from a low-frequency pattern and fundamental components extracted from a high-frequency pattern.

23. The method of claim 1, wherein the sample is a biological sample or tissue.

24. The method of claim 1, wherein the sample is a human forearm.

25. The method of claim 1, further comprising quantitative analysis of the sample.

26. The method of claim 25, wherein quantitative analysis of the sample includes quantitative analysis of tissue composition and/or changes in composition.

27. The method of claim 1, wherein the extracted images of multiple spatial frequency components are part of a multi-spectral, video-rate Spatial Frequency Domain Imaging (SFDI) system.

28. The method of claim 1, wherein the extracted images of multiple spatial frequency components are made in conjunction with a scientific-grade CMOS (sCMOS) camera.

29. The method of claim 1, wherein the extracted images of multiple spatial frequency components are made in conjunction with a digital imaging sensor.

30. The method of claim 29, wherein the digital imaging sensor includes a camera phone.

31. The method of claim 29, wherein the digital imaging sensor is a single element detector.

32. The method of claim 29, wherein the digital imaging sensor is a photodiode in a compressive sensing (CS) configuration.

33. The method of claim 1, wherein the extracted images of multiple spatial frequency components are detected by a spectrometer.

34. The method of claim 1, wherein multiple AC, non-planar spatial frequency components are extracted from the sample simultaneously.

35. The method of claim 1, wherein the sample is in vivo tissue.

36. The method of claim 1, wherein the sample is an organism.

37. The method of claim 1, wherein the sample is a plant.

38. The method of claim 1, wherein the sample is physically part of an individual.

39. The method of claim 1, wherein the sample is a turbid medium.

40. The method of claim 1, wherein the method is a component of a burn wound triage protocol.

41. The method of claim 1, wherein the method is a component of a skin cancer screening protocol.

42. The method of claim 1, wherein the method is performed in conjunction with reconstructive and/or general surgery.

43. A data and processing apparatus, comprising:
a device adapted for illuminating a sample with a binary pattern followed by a quantitative analysis of the sample.

44. The apparatus of claim 43, wherein the sample is a turbid medium.

45. The apparatus of claim 43, further comprising a projection pattern.

46. The apparatus of claim 43, wherein the projection pattern is carried by a low resolution waveguide.

47. The apparatus of claim 43, wherein the projection pattern is carried in free space.

48. The apparatus of claim 43, wherein the projection pattern is carried in a high resolution waveguide.

49. The apparatus of claim 43, wherein the projection pattern is carried in a liquid core light guide.

50. The apparatus of claim 43, wherein the projection pattern is carried by a fiber bundle.

51. The apparatus of claim 43, wherein the device is an endoscope.

52. The apparatus of claim 51, wherein the endoscope has a light source located outside of the scope component of the endoscope.

53. The apparatus of claim 43, wherein the device is adapted to extract one or more images of multiple spatial frequency components.

54. The apparatus of claim 43, wherein the device is a Spatial Frequency Domain Imaging (SFDI) system comprising a structured light illumination system configured to condense frequency information content into a frame using frequency-synthesized patterns.

55. The apparatus of claim 43, wherein quantitative analysis of the sample further comprises extracting images of multiple spatial frequency components.

56. The apparatus of claim 43, wherein quantitative analysis of the sample includes a multi-frequency synthesis and extraction (MSE) method.

57. The apparatus of claim 43, further comprising Spatial Frequency Domain Spectroscopy (SFDS).

58. The apparatus of claim 43, wherein the quantitative analysis of the sample includes fluorescence detection capabilities.

59. An optical imaging apparatus, comprising:
a structured light illumination system configured to condense frequency information content into a frame using frequency-synthesized patterns.

60. The optical imaging apparatus of claim 59, wherein the structured light illumination system is a Spatial Frequency Domain Imaging (SFDI) system.

61. The optical imaging apparatus of claim 59, wherein the sample is a turbid medium.

62. The optical imaging apparatus of claim 59, wherein the frame is part of a single Charged Coupled Device (CCD).

63. The optical imaging apparatus of claim 59, wherein the frame is part of a multi-pixel sensor array.

64. The optical imaging apparatus of claim 59, further comprising an NIR light source homogenized through an integrating rod and/or sent through a mechanical projecting device.

65. The optical imaging apparatus of claim 59, wherein the mechanical projecting device is a motorized expanding disk, non-expanding disk, fan shape, expanding ring, and/or non-expanding ring.

66. The optical imaging apparatus of claim 59, further comprising an electronic spatial light modulator.

67. The optical imaging apparatus of claim 59, further comprising a transmission and/or reflectance mask.

68. The optical imaging apparatus of claim 59, further comprising a light source with a spatial pattern.

69. The optical imaging apparatus of claim 59, further comprising Spatial Frequency Domain Spectroscopy (SFDS).

70. The optical apparatus of claim 59, further including fluorescence detection capabilities.

71. The optical apparatus of claim 59, wherein the apparatus is described in FIG. 10 herein.

72. The optical apparatus of claim 59, wherein the apparatus is described in FIG. 11 herein.

73. A method of imaging tissue, comprising:
visualizing and/or projecting a tissue sample of a subject through an optical imaging apparatus comprising a structured illumination device configure to condense frequency information content into a frame using frequency-synthesized patterns.

74. The method of claim 73, wherein the structured illumination device is a Spatial Frequency Domain Imaging (SFDI) device.

75. The method of claim 73, wherein the sample is a turbid medium.

76. The method of claim 73, wherein the frame is part of a single Charged Coupled Device (CCD).

77. The method of claim 73, wherein the frame is part of a multi-sensor pixel array.

78. The method of claim 73, wherein the optical imaging apparatus may be used to analyze physical properties of the tissue.

79. The method of claim 73, wherein physical properties includes chemical properties.

80. The method of claim 73, wherein the data acquisition speed is increased to the frame rate of a camera by using patterns.

81. The method of claim 73, wherein there is no projector chip.

82. A method of diagnosing a disease in a subject, comprising:
analyzing the physical properties of a sample from a subject using an optical imaging apparatus comprising a structured illumination device configured to condense frequency information content into a single frame using frequency-synthesized patterns; and
diagnosing the disease based on the physical properties of the sample.

83. The method of claim 82, wherein the structured illumination device is a Spatial Frequency Domain Imaging (SFDI) device.

84. The method of claim 82, wherein the single frame is a single Charged Coupled Device (CCD) frame.

85. The method of claim 82, wherein the physical properties of the sample include tissue biological function.

86. The method of claim 82, wherein the physical properties of the sample include hemodynamics and/or chemical constituents.

87. The method of claim 82, wherein the subject is human.

88. The method of claim 82, wherein the subject is an organism.

89. The method of claim 82, wherein the subject is a plant.

90. The method of claim 82, wherein the sample is a turbid medium.

91. A method of prognosing a disease and/or predicting health in a subject, comprising:
analyzing the physical properties of a sample from a subject using an optical imaging apparatus comprising a structured illumination device configured to condense frequency information content into a frame using frequency-synthesized patterns; and
determining the severity of a disease and/or predicting sample health based on the physical properties of the sample.

92. The method of claim 91, wherein the structured illumination device is a Spatial Frequency Domain Imaging (SFDI) device.

93. The method of claim 91, wherein the frame is part of a single Charged Coupled Device (CCD) frame.

94. The method of claim 91, wherein the frame is part of a multi-pixel sensor array.

95. The method of claim 91, wherein the physical properties of the sample include tissue biological function at high temporal resolution, including hemodynamics and chemical constituents.

96. The method of claim 91, further comprising analysis of time to heal from the disease.

97. The method of claim 91, wherein the subject is human.

98. The method of claim 91, further comprising treatment of the disease.

99. The method of claim 91, wherein the sample is a turbid medium.

100. A method of obtaining optical properties, and depth and fluorescence information, comprising:
illuminating and/or receiving from a sample multi-frequency patterns having arbitrary spatial frequency intensities; and
extracting a single pixel image of one or more spatial frequency components.

101. The method of claim 100, wherein the multi-frequency patterns comprises a binary square wave pattern of light using a projection pattern.

102. The method of claim 100, wherein the sample is a turbid medium.

103. A data and processing apparatus, comprising:
a device adapted for transmission of a sample with a binary pattern followed by a quantitative analysis of the sample.

104. The apparatus of claim 103, wherein the transmission includes transmission of neutrons.

105. The apparatus of claim 103, wherein the transmission includes transmission of X-Rays.

106. The apparatus of claim 103, wherein quantitative analysis of the sample includes fluorescence detection capabilities.

107. The apparatus of claim 103, wherein the sample is a turbid medium.

108. A method of evaluating tissue health in a subject, comprising:
analyzing tissue from a subject using an optical imaging apparatus comprising a structured illumination device configured to condense frequency information content into a frame using frequency-synthesized patterns to analyze the physical properties of the sample; and
evaluating tissue health based on the physical properties of the tissue.

109. The method of claim 108, wherein the structured illumination device is a Spatial Frequency Domain Imaging (SFDI) device.

110. The method of claim 108, wherein the sample is a turbid medium.

111. The method of claim 108, wherein the physical properties of the tissue include one or more of tissue biological function, chemical function, and structure.

112. The method of claim 108, wherein the frame is part of a single Charged Coupled Device (CCD) frame.

113. The method of claim 108, wherein the frame is part of a multi-pixel sensor device.

114. The method of claim 108, wherein the frame is part of a single-pixel sensor device.

115. The method of claim 108, further comprising SFD tomography.

116. The method of claim 108, wherein multi-frequency information may be extracted to generate a 3D reconstruction.

117. The method of claim 108, wherein the method is described in FIG. 9 herein.

118. An apparatus, comprising:
a transmission geometry instrument using multi-frequency patterns.

119. The apparatus of claim 118, wherein the instrument is described in FIG. 14 herein.

120. The apparatus of claim 118, wherein the instrument is described in FIG. 15 herein.
